(19) United States
(12) Patent Application Publication
(10) Pub. No.: US 2025/0318941 A1
(43) Pub. Date: Oct. 16, 2025

(54) APPARATUS AND METHODS FOR LOCKING A PROSTHETIC IMPLANT DELIVERY APPARATUS

(71) Applicant: EDWARDS LIFESCIENCES CORPORATION, Irvine, CA (US)

(72) Inventors: Roei Mayo, Irvine, CA (US); Sagi Libovitch, Sdot Yam (IL)

(73) Assignee: EDWARDS LIFESCIENCES CORPORATION, Irvine, CA (US)

(21) Appl. No.: 19/178,040

(22) Filed: Apr. 14, 2025

Related U.S. Application Data
(63) Continuation of application No. PCT/US2023/033749, filed on Sep. 26, 2023.
(60) Provisional application No. 63/380,798, filed on Oct. 25, 2022.

(51) Int. Cl. A61F 2/966; A61F 2/24

(52) U.S. Cl. CPC A61F 2/966; A61F 2/2436; A61F 2002/9665

(57) ABSTRACT

Devices and methods for locking a deliverable (e.g., a prosthetic implant, a tool, etc.) relative to a delivery apparatus are disclosed. As an example, a method of delivering a prosthetic implant can comprise inserting a delivery apparatus into a patient's vasculature, wherein the delivery apparatus comprises a first shaft, a second shaft, and an inner component disposed within a lumen of the first shaft; moving the inner component and the first shaft relative to each other; and locking the inner component and the first shaft relative to each other by applying an axial force to a shoulder of the first shaft with the second shaft.

### CROSS REFERENCE TO RELATED APPLICATION

This application is a continuation of PCT Patent Application No. PCT/US2023/033749 filed on Sep. 26, 2023, which claims the benefit of U.S. Provisional Patent Application No. 63/380,798, filed Oct. 25, 2022, each of which is incorporated by reference herein in its entirety.

### FIELD

The present disclosure relates to delivery apparatuses for prosthetic medical devices.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

### SUMMARY

Described herein are prosthetic implants, delivery apparatus, and methods for implanting prosthetic implants. The disclosed prosthetic implants, delivery apparatus, and methods can, for example, selectively lock movement of a deliverable (e.g., a prosthetic implant, a tool, an agent, or other therapy, etc.) relative to a delivery apparatus and/or selectively actuate a cutting device of a delivery apparatus by translating shafts of the delivery apparatus relative to each other. As such, the devices and methods disclosed herein can, among other things, overcome one or more of the deficiencies of typical implantation procedures for prosthetic implants and associated delivery apparatuses.

A method for delivering a prosthetic implant can comprise inserting a delivery apparatus into a patient's vasculature, moving an inner component (e.g., a tool, a prosthetic implant, etc.) and a shaft of the delivery apparatus relative to each other, and locking relative movement of the inner component and the shaft.

In some examples, a method comprises inserting a delivery apparatus into a patient's vasculature, wherein the delivery apparatus comprises a first shaft, a second shaft, and an inner component disposed within a lumen of the first shaft; moving the inner component and the first shaft relative to each other; and locking the inner component and the first shaft relative to each other by applying an axial force to a shoulder of the first shaft with the second shaft.

In some examples, a method of delivering a prosthetic implant, comprises inserting a delivery apparatus into a patient's vasculature; moving a tool within a lumen of a first shaft of the delivery apparatus relative to the first shaft, the first shaft having a first length and the lumen having a first diameter; and locking the tool within the lumen of the first shaft by moving a second shaft of the delivery apparatus relative to the first shaft in an axial direction, such that a length of the first shaft increases from the first length to a second, stretched length and a diameter of the lumen decreases from the first diameter to a second, compressed diameter.

In some examples, a method comprises one or more of the components recited in Examples 1-13 below.

A delivery apparatus for a prosthetic implant can comprise a handle and a shaft coupled to the handle.

In some examples, a delivery apparatus for a prosthetic implant, comprises a handle; a first shaft extending distally from the handle, the first shaft having a first lumen and a first durometer hardness; a second shaft disposed within the first lumen and movable relative to the first shaft, the second shaft having a second lumen and a second durometer hardness; and an inner component disposed within the second lumen, wherein the delivery apparatus is moveable between an unlocked state and a locked state, wherein in the unlocked state, the inner component is movable relative to the second shaft, wherein in the locked state, the inner component is locked relative to the second shaft, and wherein the first durometer hardness is greater than the second durometer hardness.

In some examples, a delivery apparatus for a prosthetic implant, comprises a handle; a first shaft extending distally from the handle, the first shaft comprising a first surface defining a first lumen; a second shaft extending distally from the handle, the second shaft disposed within the first lumen, the second shaft comprising a second surface defining a second lumen, wherein the first shaft and the second shaft are moveable relative to each other; and a cutting device disposed within the second lumen of the second shaft, wherein the cutting device is configured to actuate between a non-actuated state and an actuated state based on relative movement of the first shaft and the second shaft relative to the first shaft.

In some examples, a delivery apparatus comprises one or more of the components recited in Examples 14-43 below.

The above method(s) can be performed on a living animal or on a simulation, such as on a cadaver, cadaver heart, anthropomorphic ghost, simulator (e.g., with body parts, heart, tissue, etc. being simulated).

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is an elevation view of a delivery apparatus, according to one example.

FIG. 2A is a cross-sectional view of the delivery apparatus of FIG. 1 in a first configuration.

FIG. 2B is a cross-sectional view of the delivery apparatus of FIG. 1 in a second configuration.

FIG. 3A is another cross-sectional view of the delivery apparatus of FIG. 1 in the first configuration.

FIG. 3B is another cross-sectional view of the delivery apparatus of FIG. 1 in the second configuration.

FIGS. 4A-4C are cross-sectional views of the delivery apparatus of FIG. 1 having an inner shaft and inner components according to other examples.

FIG. 5A is a perspective view of a delivery apparatus for deploying a docking station, according to one example.

FIG. 5B illustrates a docking station disposed around a distal portion of the delivery apparatus of FIG. 5A.

FIG. 6A is an elevation view of a distal portion of the delivery apparatus of FIG. 5A with an outer shaft of the delivery apparatus in a retracted position.

FIG. 6B is an elevation view of a distal portion of the delivery apparatus of FIG. 5A with an outer shaft of the delivery apparatus in an extended position and cut away to show an encapsulated docking station.

FIGS. 6C-6G illustrate stages in deployment of the docking station of FIG. 5B from the delivery apparatus of FIG. 5A.

FIG. 7 is a cross-sectional view of the delivery apparatus of FIG. 1 having an inner component coupled to a docking station within a vessel of a patient, according to one example.

FIG. 8A is a cross-sectional view of a delivery apparatus according to another example in a first configuration.

FIG. 8B is a cross-sectional view of a delivery apparatus of FIG. 8A in a second configuration.

FIG. 9A is a cross-sectional view of a delivery apparatus according to another example in a first configuration.

FIG. 9B is a cross-sectional view of a delivery apparatus of FIG. 9A in a second configuration.

FIG. 10 illustrates deployment of a stent from the delivery apparatus of FIG. 8A.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (e.g., out of the patient's body), while distal motion of the device is motion of the device away from the user and toward the implantation site (e.g., into the patient's body). The terms “longitudinal” and “axial” refer to an axis extending in the proximal and distal directions, unless otherwise expressly defined.

As used herein, “e.g.” means “for example,” and “i.e.” means “that is.”

### Introduction to the Disclosed Technology

Described herein are examples of a steerable delivery apparatus (sometimes referred to as a steerable catheter) that can be used to navigate a subject's vasculature to deliver an implantable, expandable medical device (e.g., a prosthetic heart valve, a stent, etc.), tools, agents, or other therapy to a location within the body of a subject. Examples of procedures in which the steerable catheters are useful include neurological, urological, gynecological, fertility (e.g., in vitro fertilization, artificial insemination), laparoscopic, arthroscopic, transesophageal, transvaginal, transvesical, transrectal, and procedures including access in any body duct or cavity. Particular examples include placing implants, including stents, grafts, embolic coils, and the like; positioning imaging devices and/or components thereof, including ultrasound transducers; and positioning energy sources, for example, for performing lithotripsy, RF sources, ultrasound emitters, electromagnetic sources, laser sources, thermal sources, and the like.

The delivery apparatuses described herein can be configured to lock movement of a deliverable (e.g., a prosthetic implant, a tool, an agent, or other therapy, etc.) or at least a portion of a deliverable relative to the delivery apparatus. As a result, a positioning of the deliverable can be easily maintained relative to the delivery apparatus and/or adjusted during an implantation procedure in a streamlined manner. In some examples, this can increase an efficiency of the implantation procedure.

### Examples of the Disclosed Technology

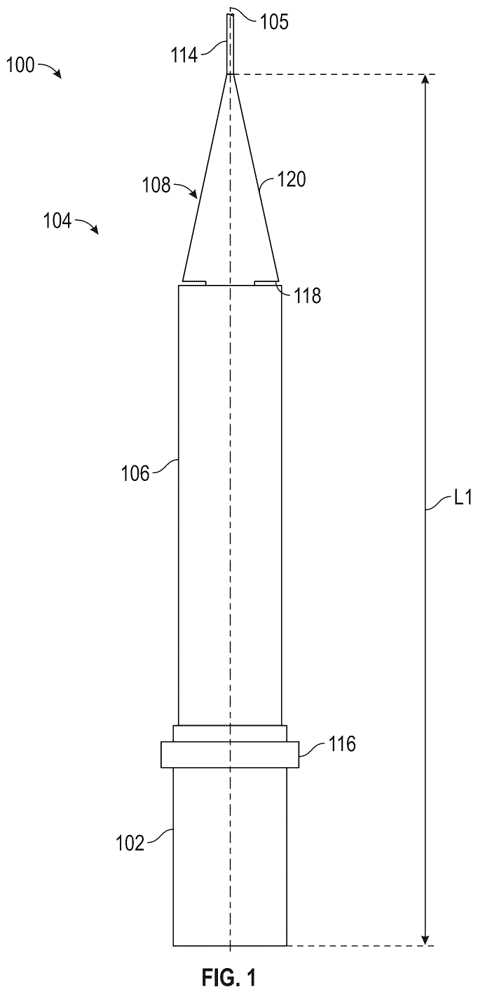

FIGS. 1-3B illustrate an exemplary delivery apparatus 100 that can be used to deliver a deliverable such as a prosthetic implant (e.g., a stent, a docking device, a valve, etc.) and/or a tool (e.g., a guidewire, a stylet, a snare, a gripping device, a coil, etc.) to an implantation location, for example, within a patient's vasculature. The delivery apparatus 100 generally includes a handle 102 and a shaft assembly 104 coupled to the handle 102 and extending distally from the handle 102. The shaft assembly 104 can include a central longitudinal axis 105. The shaft assembly 104 includes an inner shaft 106 and an outer shaft 108. The inner shaft 106 includes a first lumen 110 defined by an inner surface thereof. The outer shaft 108 includes a second lumen 112 defined by an inner surface thereof. The inner shaft 106 extends through the second lumen 112 of the outer shaft 108.

In some examples, as shown in FIGS. 1-3B, an inner component 114 can be disposed within the first lumen 110. The inner component 114 can comprise any deliverable, including a prosthetic implant (e.g., a stent, a docking device, a valve, etc.), a tool (e.g., a guidewire, a stylet, a snare, a gripping device, a coil, etc.), or the like. In the illustrated example, the inner component 114 is a guidewire. As described in more detail below, the delivery apparatus 100 can be configured to selectively lock the inner component 114 relative to the delivery apparatus 100, such that the inner component 114 cannot move (e.g., rotate, translate, etc.) relative to the delivery apparatus 100. In other words, when locked, the inner component 114 and the delivery apparatus 100 move together.

The inner shaft 106 and the outer shaft 108 can be moveable relative to each other. For example, the inner shaft 106 and the outer shaft 108 can be axially moveable and/or rotationally moveable relative to each other. In some examples, the handle 102 can comprise input devices to manipulate the shaft assembly 104. For example, as depicted, the handle 102 can include a knob 116 configured to move the inner shaft 106 and the outer shaft 108 relative to each other. For example, the outer shaft 108 can be coupled to the knob 116, such that rotation of the knob 116 in a first direction (e.g., clockwise) moves the outer shaft 108 relative to the inner shaft 106 in a first direction (e.g., a distal direction). Rotation of the knob 116 in a second direction (e.g., counter-clockwise) can move the outer shaft 108 relative to the inner shaft 106 in a second direction (e.g., in a proximal direction). In some examples, the knob 116 can be coupled to the inner shaft 106 to move the inner shaft 106 relative to the outer shaft 108.

The inner shaft 106 can comprise a shoulder 118. The outer shaft 108 is disposed proximal to the shoulder 118. In some examples, the shoulder 118 is disposed at or adjacent to a distal end of the inner shaft 106. The shoulder 118 can have an outer diameter that is larger than the second lumen 112 of the outer shaft 108, as depicted. In some examples, the outer diameter of the shoulder 118 can be less than, equal to, or larger than an outer diameter of the outer shaft 108. In some examples, the shoulder 118 can be part of a nosecone 120 (FIG. 1) coupled to a distal end of the inner shaft 106. Specifically, as shown, the shoulder 118 is disposed at a proximal (or widest) end of the nosecone 120 and the nosecone 120 is tapered in the distal direction. In some examples, not shown, the shoulder 118 can be part of a flange having a constant outer diameter (e.g., a flange that is not tapered, etc.). In some examples, as depicted, the inner shaft 106 and the nosecone 120 (or flange, etc.) can be formed as a single, monolithic piece. In some examples, not shown, the inner shaft 106 and the nosecone 120 can be separately formed pieces that are coupled together.

In some examples, the shoulder 118 can limit axial movement of the inner shaft 106 and the outer shaft 108 relative to each other. For example, when the outer shaft 108 is translated distally relative to the inner shaft 106 along axis 105 (or the inner shaft 106 is translated proximally relative to the outer shaft 108 along axis 105), a distal end 108d of the outer shaft 108 can abut the shoulder 118. In some examples, if the outer shaft 108 is translated distally relative to the inner shaft 106 by an additional amount (or the inner shaft 106 is translated proximally relative to the outer shaft 108 by an additional amount) (e.g., after the distal end 108d is already abutting the shoulder 118), the outer shaft 108 can apply an axially directed force F_{A} to the shoulder 118 that is sufficient to elastically deform the inner shaft 106. Specifically, the axial force F_{A} is parallel to axis 105 and can elongate (e.g., increase) a length of the inner shaft 106 in an axial direction and compress (e.g., decrease) a diameter of the first lumen 110 in a radial direction. The magnitude of the axial force F_{A} can be proportional to the amount of deformation of the inner shaft 106 (e.g., the elongation of the inner shaft 106 and/or the compression of the first lumen 110).

In some examples, the inner shaft 106 and the outer shaft 108 can comprise materials having different hardness properties and/or different tensile strengths. The different properties of the materials can enable the inner shaft 106 to elongate and the first lumen 110 to compress when the axial force F_{A} is applied to the shoulder 118 (e.g., by the outer shaft 108). Specifically, the amount of deformation of the inner shaft 106 and/or the force F_{A} required to deform the inner shaft 106 can depend on the relative properties of the inner shaft 106 and the outer shaft 108. For example, the durometer hardness of the outer shaft 108 can be greater than the durometer hardness of the inner shaft 106. In some examples, the durometer hardness of the outer shaft 108 can be at least 1.5 times greater than the durometer hardness of the inner shaft 106. The tensile strength of the outer shaft 108 can be greater than the tensile strength of the inner shaft 106. In some examples, the tensile strength of the outer shaft 108 can be at least three times greater than the tensile strength of the inner shaft 106.

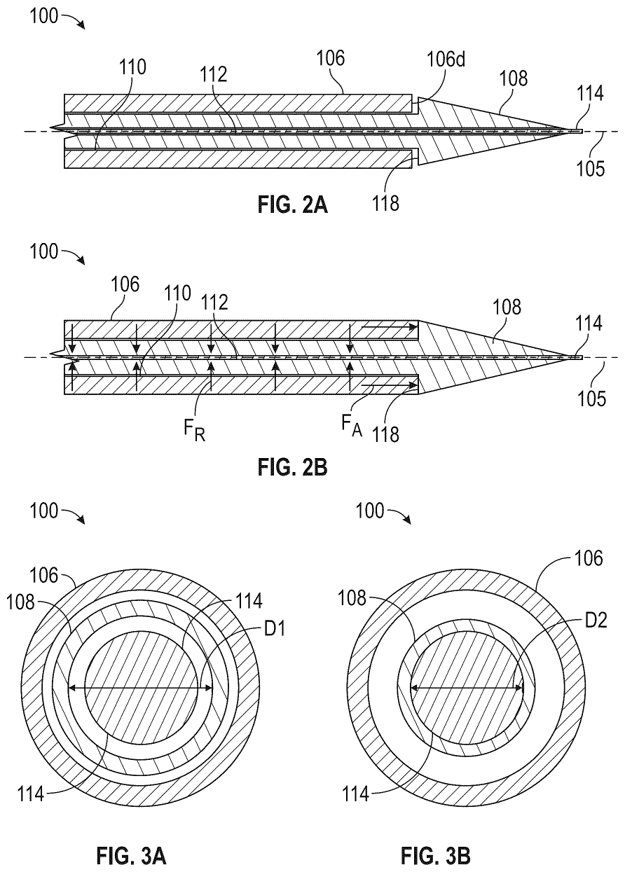

The delivery apparatus 100 can be transitioned between an unlocked state and a locked state by translating the inner shaft 106 and the outer shaft 108 relative to each other. FIGS. 1-2A and 3A illustrate the delivery apparatus 100 in the unlocked state. FIGS. 2B and 3B illustrate the delivery apparatus 100 in the locked state. In the unlocked state, the inner component 114 is moveable relative to the inner shaft 106 (and therefore the delivery apparatus 100). For example, the inner component 114 is able to move axially within the first lumen 110 and/or rotate within the first lumen 110 relative to the inner shaft 106. In the locked state, the inner component 114 is locked (e.g., immovable) relative to the inner shaft 106 (and therefore the delivery apparatus 100). For example, the inner component 114 is not able to move axially within the first lumen 110 and/or rotate within the first lumen 110.

In the unlocked state (FIGS. 1-2A, 3A), the outer shaft 108 applies an axial force (e.g., no force, a nominal force, etc.) to the shoulder 118 that is insufficient to deform the inner shaft 106 (e.g., elongate the inner shaft 106 and compress the first lumen 110). As such, in the unlocked state, the distal end 108d of the outer shaft 108 can either be spaced apart from the shoulder 118 or abut the shoulder 118 without applying sufficient axial force. For example, in some instances (e.g., when the outer shaft 108 abuts the shoulder 118 in the unlocked state), a nominal force (e.g., a force less than axial force F_{A}) may be applied to the shoulder 118 that does not deform the inner shaft 106 to the locked state.

In the unlocked state, the inner shaft 106 comprises a first (e.g., unstretched) length L1 (FIG. 1) and the first lumen 110 comprises a first (e.g., uncompressed) diameter D1 (FIG. 3A). As shown in FIG. 3A, the first diameter D1 is greater than an outer diameter of the inner component 114. In this way, the inner component 114 can move (e.g., translate, rotate, etc.) relative to the inner shaft 106 within the first lumen 110.

To transition from the unlocked state (FIGS. 1-2A, 3A) to the locked state (FIGS. 2B, 3B), the inner shaft 106 and the outer shaft 108 are translated relative to each other (e.g., by rotating knob 116) along axis 105. In the locked state, the outer shaft 108 applies a sufficient axial force F_{A} to the shoulder 118 to deform the inner shaft 106 (e.g., elongate the inner shaft 106 and compress the first lumen 110). For example, the outer shaft 108 can be translated distally relative to the inner shaft 106 (or the inner shaft 106 can be translated proximally relative to the outer shaft 108) until the outer shaft 108 applies the sufficient axial force F_{A} to the shoulder 118, thus achieving the locked state. As such, in the locked state, the distal end 108d of the outer shaft 108 abuts or contacts the shoulder 118 while applying a sufficient axial force F_{A} to deform the inner shaft 106.

In the locked state, the inner shaft 106 comprises a second (e.g., stretched) length (not shown) and the first lumen 110 comprises a second (e.g., compressed) diameter D2 (FIG. 3B). As shown, the second, stretched length of the inner shaft 106 is greater than the first, unstretched length L1. The first and second lengths can be an entire length of the inner shaft 106 (as L1 is shown in FIG. 1) or a portion of the inner shaft 106. For example, the first and second lengths can be measured along axis 105 between a distal end of the handle 102 and the shoulder 118. As shown in FIGS. 3A-3B, the first, uncompressed diameter D1 is greater than the second, compressed diameter D2. As described above, the magnitude of the axial force F_{A} can be proportional to the elongation of the inner shaft 106 and the compression of the first lumen 110.

As shown in FIG. 3B, the second diameter D2 is equal to the outer diameter of the inner component 114, such that the first lumen 110 contacts the inner component 114 (e.g., compresses against the inner component 114). For example, the inner component 114 can limit the ability of the first lumen 110 to compress further in the radial direction. In this way, in the locked state, the inner shaft 106 applies a compressive, radial force F_{R} to the inner component 114. The radial force F_{R} can be sufficient to lock the inner component 114 relative to the inner shaft 106 (e.g., such that the inner component 114 cannot move axially and/or rotationally relative to the inner shaft 106 within the first lumen 110). When the radial force F_{R} is applied, friction between the inner component 114 and the inner shaft 106 can prevent the relative movement therebetween. In some examples, the radial force F_{R} can be proportional to the axial force F_{A}.

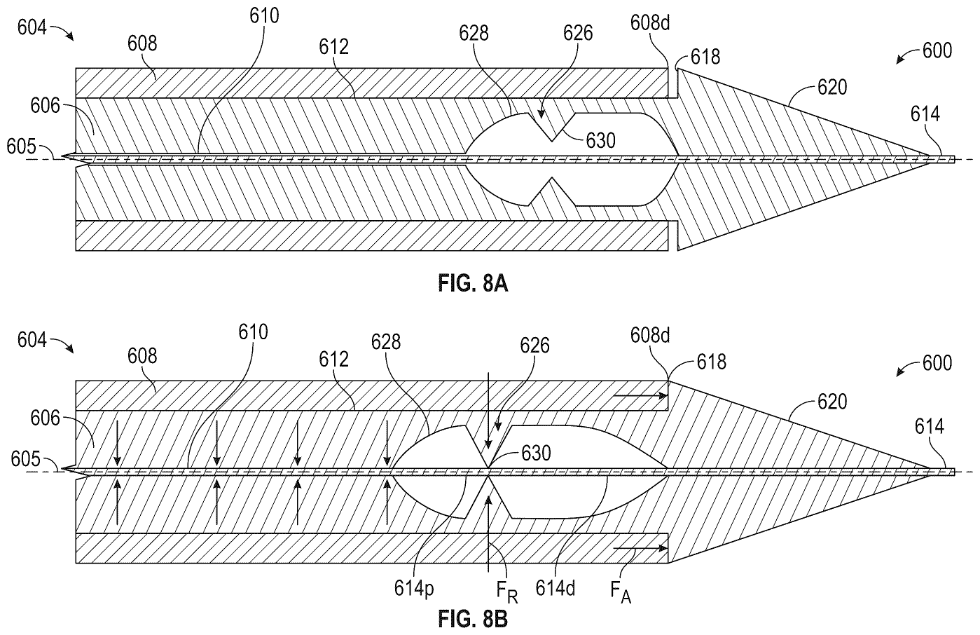

As shown, the radial force F_{R} can be distributed along a length of the inner shaft 106. For example, the radial force F_{R} can be distributed (e.g., evenly distributed) along a portion of the inner shaft 106 comprising the same material properties (e.g., durometer hardness, tensile strength, etc.). In this way, the radial force F_{R} applied by the inner shaft 106 can compress the inner component 114 within the first lumen 110 without application of a localized force. As such, the radial force F_{R} can be distributed in a manner that locks the inner component 114 relative to the inner shaft 106 while preventing deformation and/or damage to the inner component 114. In some examples (as shown in FIGS. 8A-10), an inner shaft can be configured with an inner structure (e.g., a cutting device) disposed within a lumen to localize at least some of the radial force F_{R} applied to an inner component, as desired (e.g., to cut the inner component).

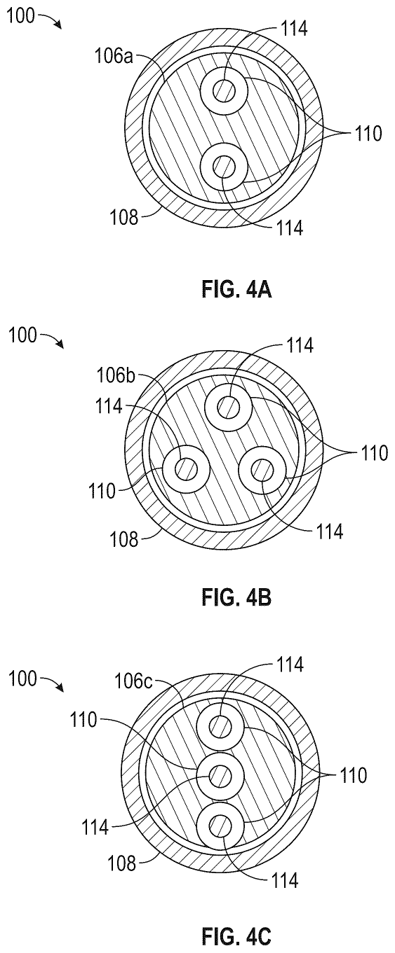

In some examples, as shown in FIGS. 4A-4C, the inner shaft of the delivery apparatus 100 can comprise multiple first lumens 110, such that multiple inner components 114 can be disposed within the first lumens 110 of the inner shaft. For example, FIG. 4A illustrates an inner shaft 106a having two first lumens 110, with an inner component 114 disposed in each lumen. FIGS. 4B-4C illustrate inner shafts 106b and 106c having three first lumens 110, with an inner component 114 disposed in each first lumen 110. The first lumens 110 of the inner shaft 106b are circumferentially spaced apart (e.g., evenly spaced apart by 120 degrees, etc.). The first lumens 110 of the inner shaft 106c are disposed in a line (e.g., with a central first lumen 110 disposed radially between the other two first lumens 110). In some examples, different compressive forces (e.g., different radial forces F_{R}) can be applied to inner components 114 disposed within different ones of the first lumens 110 (e.g., based on the configuration of the first lumens 110), such that different axial forces (e.g., axial force F_{A}) applied to the shoulder 118 are required to lock the inner components 114 relative to the inner shaft. For example, different compressive forces may be applied by inner shafts having asymmetrically positioned lumens. The delivery apparatus 100 can include any of the inner shafts described herein (e.g., inner shaft 106, shafts 106a-106c, etc.). While some configurations of inner shafts having multiple lumens are shown, the delivery apparatus 100 can include inner shafts having multiple lumens disposed in different arrangements. Although not shown, in some examples, an inner shaft can include more than three lumens.

In some instances, the radial spacing between components (e.g., between inner component and first lumen, between outer shaft and inner shaft, etc.) in FIGS. 3A-4C has been modified for purposes of illustration. For example, in the unlocked state of FIGS. 3A and 4A-4C, the spacing between the inner component(s) 114 and the lumen(s) 110 appears exaggerated for illustration purposes.

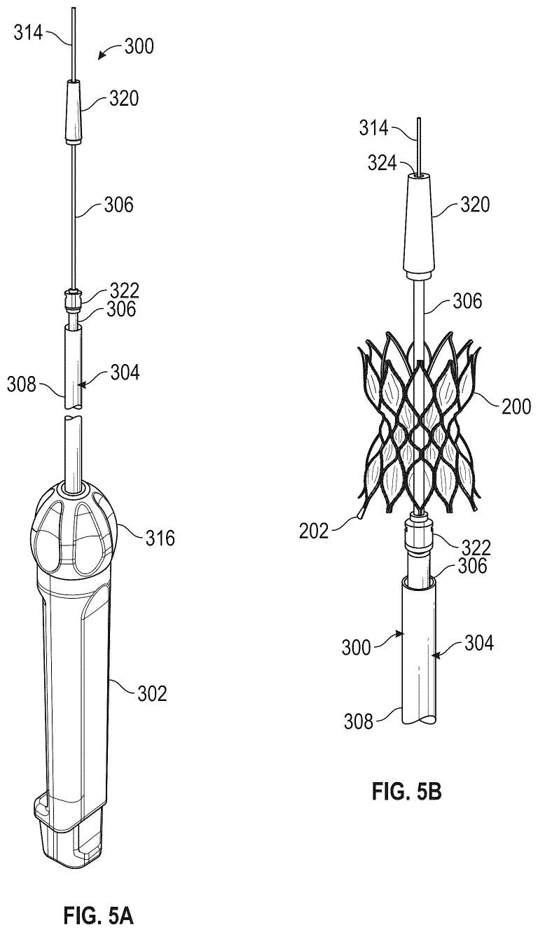

FIG. 5A illustrates an exemplary delivery apparatus 300 that can be used to deliver a deliverable such as a prosthetic implant (e.g., a stent, a docking device, a valve, etc.) to an implantation location. The delivery apparatus 300 generally includes a handle 302 and a shaft assembly 304 coupled to the handle 302 and extending distally from the handle 302. The shaft assembly 304 includes an inner shaft 306 and an outer shaft 308. The inner shaft 306 extends through a lumen of the outer shaft 308. As described in more detail below, the inner shaft 306 and the outer shaft 308 can be moveable relative to each other to lock a component (e.g., guidewire 314) within a lumen of the inner shaft 306, similar to delivery apparatus 100.

In the example illustrated by FIG. 5A, a frame connector 322 is coupled to the inner shaft 306. A docking station 200 can be disposed around a portion of the inner shaft 306 extending distally from the frame connector 322, as shown in FIG. 5B. In one example, the frame connector 322 includes one or more recesses that can receive one or more connector tabs 202 at the proximal end of the docking station 200 and thereby axially restrain the docking station 200. Additional examples of frame connectors and docking stations are disclosed in International Application No. PCT/US2022/018093, which is incorporated by reference herein in its entirety.

A nosecone 320 can be attached to a distal end of the inner shaft 306. The nosecone 320 includes a central opening 324 for receiving a guidewire 314. As such, a proximal end of the guidewire 314 can be inserted into the central opening 324 and through the inner shaft 306, and a distal end portion of the delivery apparatus 300 can be advanced over the guidewire 314 through a patient's vasculature and to an implantation location. The guidewire 314 can pass through the nosecone 320 into a lumen of the inner shaft 306 during advancing of the delivery apparatus 300 through a patient's vasculature.

The handle 302 can be operated to move the outer shaft 308 relative to the inner shaft 306, generally between an extended position and a retracted position, for example, using knob 316. The handle 302 can be extended to slide the outer shaft 308 over the frame connector 322 and over any docking station coupled to the frame connector 322 to encapsulate the docking station within the outer shaft 308. As the outer shaft 308 slides over the docking station 200, the outer shaft 308 can compress the docking station 200 such that the docking station is encapsulated within the outer shaft 308 in the compressed state. In an extended position, a distal end of the outer shaft 308 can abut a shoulder 318 of the nosecone 320 such that there are no gaps in the delivery assembly. Additionally (or alternatively), a crimping device can be used to radially compress the docking station such that it can be inserted into the outer shaft of the delivery apparatus.

As described in more detail below, the retracted and extended positions can be unlocked states of the delivery apparatus 300 (e.g., the outer shaft 308 does not contact the shoulder 318 and/or does not apply a sufficient axial force F_{A} (FIG. 5G) to the shoulder 318 to deform the inner shaft 306). In some examples, the extended position can be a locked state of the delivery apparatus 300 (e.g., when the distal end of the outer shaft 308 applies a sufficient axial force F_{A} to the shoulder 318 to deform the inner shaft 306).

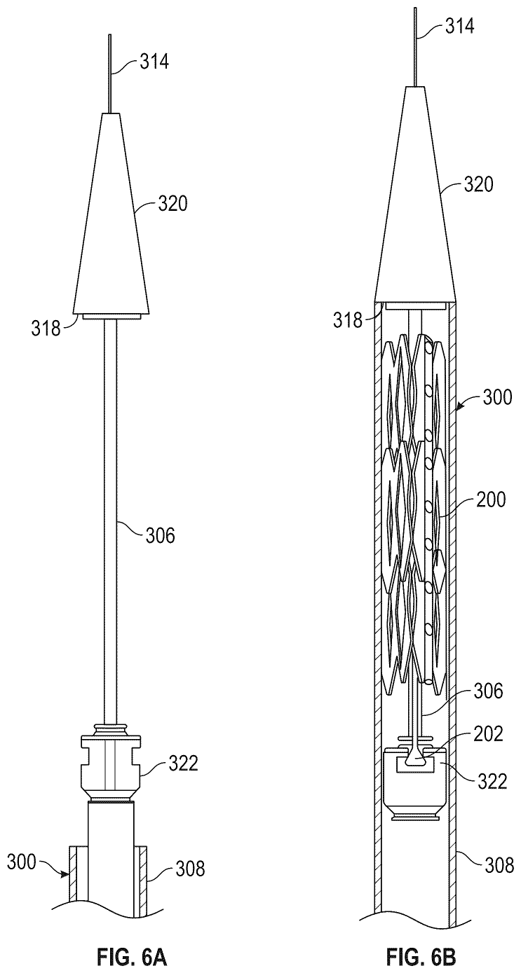

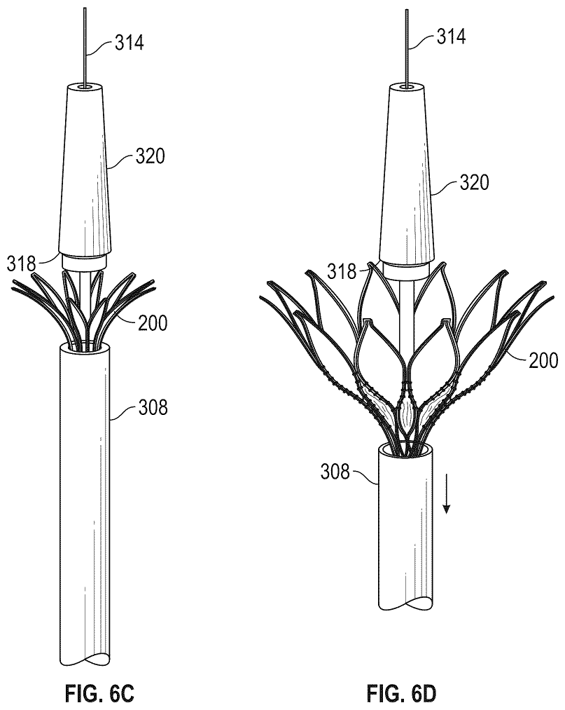

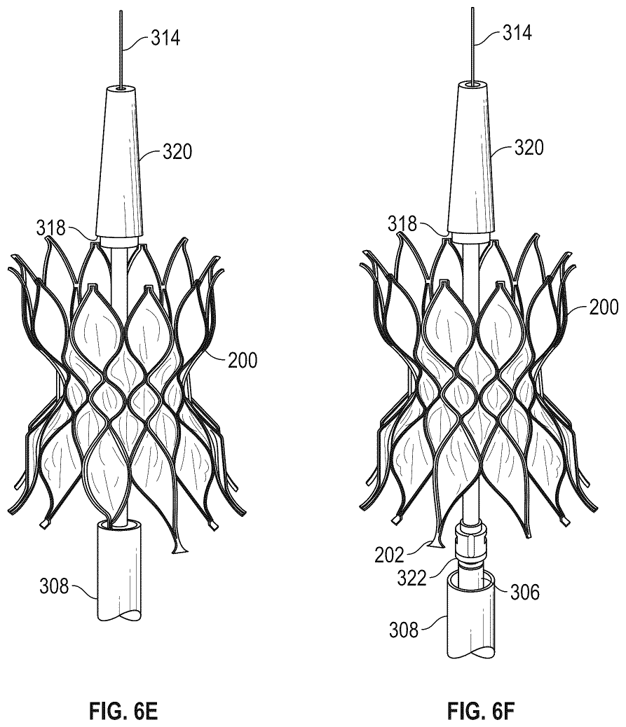

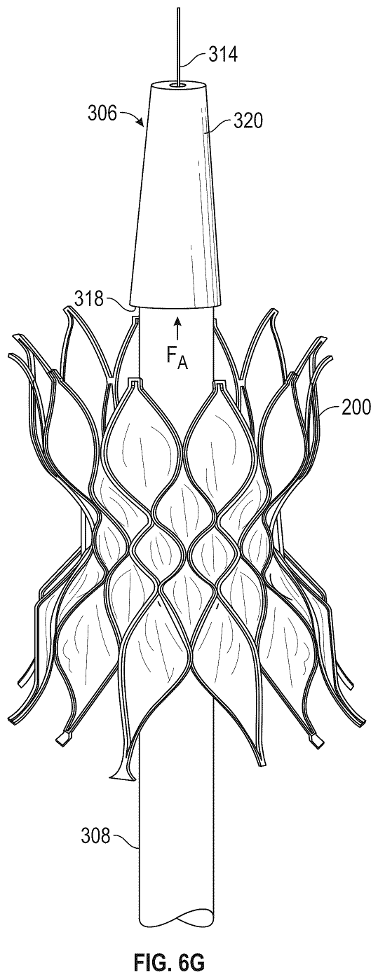

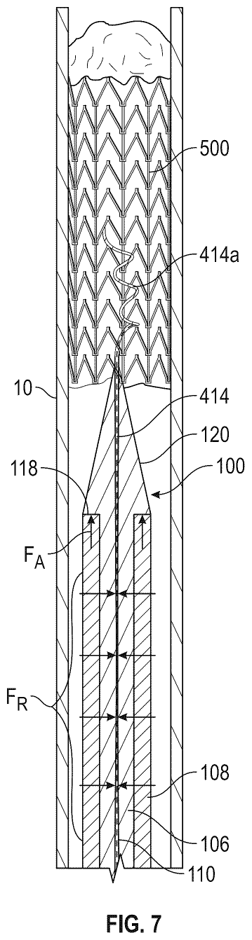

FIGS. 6A-7D illustrate a method of deploying a docking station at an implantation location within an anatomy. For purposes of illustration, the patient's anatomy is omitted. In FIG. 6A, the method includes retracting the outer shaft 308 by the handle of the delivery apparatus to allow loading of the docking station 200 onto the inner shaft 306. In FIG. 6B, the method includes disposing the docking station 200 around the inner shaft 306 and engaging each of the connector tabs 202 of the docking station 200 with the frame connector 322. The method also includes positioning the outer shaft 308 over the docking station such that the docking station is encapsulated therein. This can be accomplished by manipulating the handle 302 of the delivery apparatus 300 (e.g., by rotating knob 316, etc.). As shown in FIG. 6B, the distal end of the outer shaft 308 abuts the shoulder 318 (e.g., the proximal end of the nosecone 320). Although the distal end of the outer shaft 308 abuts the shoulder 318, the delivery apparatus 300 can be in an unlocked state (as described above in connection with delivery apparatus 100), such that the delivery apparatus 300 is permitted to translate relative to the guidewire 314 (e.g., along the guidewire 314). The method includes inserting the delivery apparatus 300, from the nosecone 320 end, into a patient's vasculature and advancing the delivery apparatus 300 through the patient's vasculature to the implantation location.

At the implantation location, the method includes retracting the outer shaft 308 by the handle of the delivery apparatus to expose the docking station 200. FIGS. 6C-6F show different stages of retracting the outer shaft 308. As can be seen, in cases where the docking station 200 is self-expanding, the docking station 200 gradually emerges from the outer shaft 308 and gradually expands from the compressed state as the outer shaft 308 is retracted. When the outer shaft 308 is sufficiently retracted, the connector tabs 202 disengage from the frame connector 322. Once the docking station 200 is disengaged from the frame connector 322, the docking station 200 can radially expand to engage the anatomy.

As shown in FIG. 6G, at various times throughout the procedure (e.g., after the docking station 200 is implanted at the implantation location, etc.), the delivery apparatus can be transitioned between the unlocked state (FIGS. 6A-6F) and a locked state (FIG. 6G). The unlocked and locked states of the delivery apparatus 300 are the same as the unlocked and locked states of the delivery apparatus 100, as described above. Specifically, to transition the delivery apparatus 300 to the locked state, the inner shaft 306 and the outer shaft 308 can be translated relative to each other (e.g., using knob 316 (FIG. 5A)) to lock the inner shaft 306 (and therefore the delivery apparatus 300) relative to the guidewire 314. For example, as described above in connection with delivery apparatus 100, in the locked state, the outer shaft 308 can abut the shoulder 318 and apply a sufficient axial force F_{A} to deform the inner shaft 306, such that the inner shaft 306 elongates in an axial direction (e.g., parallel to the axial force F_{A}) and compresses radially inward around the guidewire 314, applying a radial force (not shown) to the guidewire 314. In this way, the guidewire 314 can be locked relative to the inner shaft 306 (and therefore the delivery apparatus 300), such that the guidewire 314 and the inner shaft 306 cannot move (e.g., rotate and/or translate) relative to each other.

As described above, the compressive radial force applied by the inner shaft 306 to the guidewire 314 in the locked state can be distributed along a length of the inner shaft 106. For example, the radial force can be distributed (e.g., evenly distributed) along a portion of the inner shaft 106 comprising the same material properties (e.g., durometer hardness, tensile strength, etc.). In some examples, the radial force can be applied to the guidewire 314 by the inner shaft 306 along a length of the inner shaft 306, including at a location proximal to the frame connector 322 (see FIGS. 5A-5B).

In some examples, transitioning a delivery apparatus between an unlocked state and a locked state relative to a deliverable such as a tool (e.g., a guidewire, a stylet, a snare, a gripping device, a coil, etc.) can be useful for positioning and/or repositioning the tool relative to a patient's anatomy and/or relative to a prosthetic implant disposed within the patient's anatomy. For example, FIG. 7 illustrates the delivery apparatus 100 in the locked state relative to a patient's vasculature 10. As shown, a tool 414 is disposed within the inner shaft 106. In the locked state, the outer shaft 108 abuts the proximal end of the nosecone 120 (e.g., shoulder 118) and applies a sufficient axial force F_{A} to the nosecone 120 to elongate the inner shaft 106 and radially compress the inner shaft 106. As shown, a radial force F_{R} is applied to the tool 414 to lock the tool 414 relative to the inner shaft 106 (and therefore relative to the delivery apparatus 100), such that the tool 414 cannot rotate and/or translate relative to the inner shaft 106).

In some examples, as shown, the tool 414 can be used to retrieve and/or manipulate objects (e.g., prosthetic implant 500) in the body of a patient (e.g., in the patient's vasculature 10). For example, a distal end portion 414a can be used to couple the tool 414 to the prosthetic implant 500. As shown, the distal end portion 414a of the tool 414 comprises a coil. In some examples, the tool 414 can be advanced from a retracted configuration, with the distal end portion 414a of the tool 414 disposed within the lumen 110 of the inner shaft 106 (not shown), to a deployed configuration with the distal end portion 414a external and distal to the lumen 110 of the inner shaft 106, as shown in FIG. 7. In the retracted configuration, the lumen 110 can retain the distal end portion 414a in a straightened or uncoiled position.

The tool 414 can be transitioned between the retracted configuration and the deployed configurations when the delivery apparatus 100 is in an unlocked state. For example, the tool 414 can be moved (e.g., translated, rotated, etc.) relative to the inner shaft 106 when the delivery apparatus is in the unlocked state. Once the tool 414 is in the deployed configuration, the distal end portion 414a of the tool 414 can be coupled to the prosthetic implant 500. In some examples, to couple the tool 414 to the prosthetic implant 500, the tool 414 can be rotated relative to the inner shaft 106 within the lumen 110, for example, to engage the coil through openings within a frame of the prosthetic implant 500. The distal end portion 414a can be coupled to the prosthetic implant 500 in other manners, for example, depending on the configuration of the tool and/or the prosthetic implant.

In some examples, the delivery apparatus 100 can be transitioned to the locked state prior to coupling the tool 414 to the prosthetic implant 500. For example, after the inner shaft 106 and the outer shaft 108 are moved relative to each other to lock the tool 414 relative to the delivery apparatus 100, the delivery apparatus 100 can be manipulated (e.g., rotated, etc.) relative to the patient's vasculature 10 and/or relative to the prosthetic implant 500, such that the distal end portion 414a of the tool 414 is engaged with the prosthetic implant 500 (e.g., through the openings of the prosthetic implant 500).

When the delivery apparatus 100 is in the locked state and the tool 414 is coupled to the prosthetic implant 500, as shown in FIG. 7, the delivery apparatus 100 can be manipulated (e.g., translated, etc.) relative to the patient's vasculature 10 to adjust a positioning of the prosthetic implant 500 within the patient's vasculature 10. For example, the prosthetic implant 500 can be moved from an initial implant location to a location that is distal or proximal to the initial implant location and/or can be rotated relative to the patient's vasculature 10 by manipulation of the delivery apparatus 100. As a result, a positioning of the prosthetic implant 500 can be easily adjusted relative to the delivery apparatus 100 during an implantation procedure in a streamlined manner. In some examples, this can increase an efficiency of the implantation procedure.

After the prosthetic implant 500 is positioned in a desired location, the tool 414 can be uncoupled (e.g., detached) from the prosthetic implant 500. Once uncoupled, the tool 414 and the delivery apparatus 100 can be removed from the patient's vasculature 10. The delivery apparatus 100 can be in the locked state during removal, such that the delivery apparatus 100 and the tool 414 are removed together.

In some examples, transitioning a delivery apparatus between an unlocked state and a locked state relative to a deliverable such as a prosthetic implant (e.g., a stent, etc.) can be useful for deploying the prosthetic implant from the delivery apparatus. Specifically, in some examples, an inner shaft of a delivery apparatus can include a cutting device (e.g., a knife, a blade, a wedge, etc.) disposed therein that can be actuated by transitioning the delivery apparatus between the unlocked and locked states. For example, actuating the cutting device can detach the deliverable from the delivery apparatus for implantation within a patient's vasculature. As such, in some examples, the unlocked state of the delivery apparatus can also be referred to as a non-actuated state and the locked state of the delivery apparatus can also be referred to as an actuated state.

FIGS. 8A-8B illustrate an exemplary delivery apparatus 600 that can be used to deliver a deliverable such as a prosthetic implant (e.g., a stent, etc.) to an implantation location, for example, within a patient's vasculature. The delivery apparatus 600 can be similar to the delivery apparatus 100 and/or delivery apparatus 300, although the delivery apparatus 600 includes a cutting device (e.g., a knife, a blade, a wedge, etc.), as described in more detail below. For example, similar to the delivery apparatuses described herein, the delivery apparatus 600 can include a handle and a shaft assembly 604 coupled to the handle and extending distally from the handle. The shaft assembly 604 can include a central longitudinal axis 605. The shaft assembly 604 includes an inner shaft 606 and an outer shaft 608. The inner shaft 606 includes a first lumen 610 defined by an inner surface thereof. The outer shaft 608 includes a second lumen 612 defined by an inner surface thereof. The inner shaft 606 extends through the second lumen 612 of the outer shaft 608.

The inner shaft 606 can comprise a shoulder 618. The outer shaft 608 is disposed proximal to the shoulder 618. In some examples, the shoulder 618 is disposed at or adjacent to a distal end of the inner shaft 606. The shoulder 618 can have an outer diameter that is larger than the second lumen 612 of the outer shaft 608, as depicted. In some examples, the outer diameter of the shoulder 618 can be smaller than, equal to, or larger than an outer diameter of the outer shaft 608. In some examples, the shoulder 618 can be part of a nosecone 620 coupled to a distal end of the inner shaft 606. Specifically, as shown, the shoulder 618 is disposed at a proximal (or widest) end of the nosecone 620 and the nosecone 620 is tapered in the distal direction. In some examples, not shown, the shoulder 618 can be part of a flange having a constant outer diameter (e.g., a flange that is not tapered, etc.). In some examples, as depicted, the inner shaft 606 and the nosecone 620 (or flange, etc.) can be formed as a single, monolithic piece. In some examples, not shown, the inner shaft 606 and the nosecone 620 can be separately formed pieces that are coupled together.

The inner shaft 606 and the outer shaft 608 can be moveable relative to each other. For example, the inner shaft 606 and the outer shaft 608 can be axially moveable and/or rotationally moveable relative to each other (e.g., by manipulating an input device on a handle of the delivery apparatus 600, etc.). In some examples, the shoulder 618 can limit axial movement of the inner shaft 606 and the outer shaft 608 relative to each other. For example, when the outer shaft 608 is translated distally relative to the inner shaft 606 along axis 605 (or the inner shaft 606 is translated proximally relative to the outer shaft 608 along axis 605), a distal end 608d of the outer shaft 608 can abut the shoulder 618. In some examples, if the outer shaft 608 is translated distally relative to the inner shaft 606 by an additional amount (or the inner shaft 606 is translated proximally relative to the outer shaft 608 by an additional amount) (e.g., after the distal end 608d is already abutting the shoulder 618), the outer shaft 608 can apply an axially directed force F_{A} to the shoulder 618 that is sufficient to elastically deform the inner shaft 606. Specifically, the axial force F_{A} is parallel to axis 605 and can elongate (e.g., increase) a length of the inner shaft 606 in an axial direction and compress (e.g., decrease) a diameter of the first lumen 610 in a radial direction. The magnitude of the axial force F_{A} can be proportional to the amount of deformation of the inner shaft 606 (e.g., the elongation of the inner shaft 606 and/or the compression of the first lumen 610).

In some examples, the inner shaft 606 and the outer shaft 608 can comprise materials having different hardness properties and/or different tensile strengths. The different properties of the materials can enable the inner shaft 606 to elongate and the first lumen 610 to compress when the axial force F_{A} is applied to the shoulder 618 (e.g., by the outer shaft 608). Specifically, the amount of deformation of the inner shaft 606 and/or the force F_{A} required to deform the inner shaft 606 can depend on the relative properties of the inner shaft 606 and the outer shaft 608. For example, the durometer hardness of the outer shaft 608 can be greater than the durometer hardness of the inner shaft 606. In some examples, the durometer hardness of the outer shaft 608 can be at least 1.5 times greater than the durometer hardness of the inner shaft 606. The tensile strength of the outer shaft 608 can be greater than the tensile strength of the inner shaft 606. In some examples, the tensile strength of the outer shaft 608 can be at least three times greater than the tensile strength of the inner shaft 606.

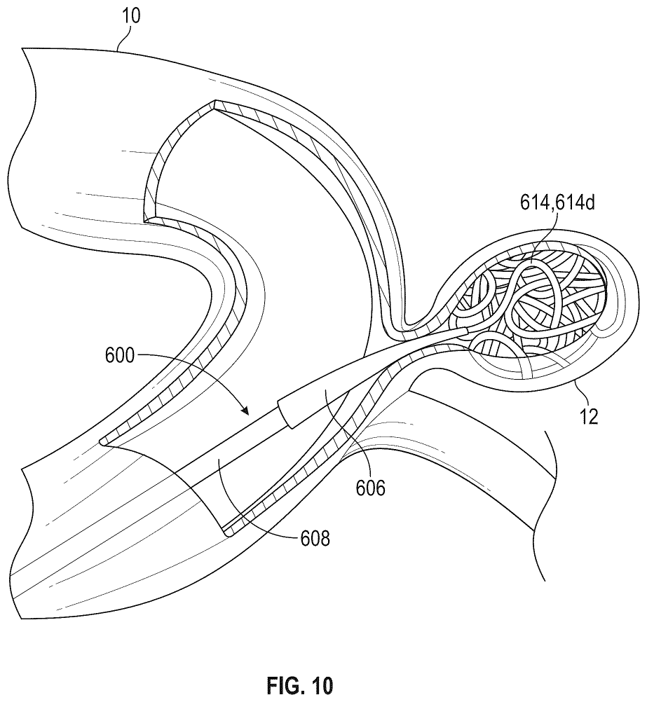

In some examples, as shown in FIGS. 8A-8B, a stent 614 can be disposed within the first lumen 610. It should be appreciated that the delivery apparatus 600 can be used with other inner components and/or deliverables that may be cut with cutting device 626. In the illustrated example, the stent 614 is a coil stent (e.g., an annular coil stent, a ganglion-shaped stent, etc.). When the stent 614 is advanced distally relative to the inner shaft 606 (e.g., such that the stent 614 is not retained within the inner shaft 606), the stent 614 can coil, for example, within an aneurysm 12 of a patient's vasculature 10 (FIG. 10). In some examples, the stent 614 can be formed from a shape-memory material (e.g., Nitinol, etc.).

As shown in FIGS. 8A-8B, the cutting device 626 can be coupled to the inner shaft 606 and disposed within the first lumen 610. As shown, the cutting device 626 can be disposed within a cutting region 628 of the inner shaft 606. The cutting region 628 of the first lumen 610 can have a larger diameter than other regions of the first lumen 610 (e.g., the region of the first lumen 610 positioned within the nosecone 620, a proximal region of the first lumen 610, etc.). The relatively larger diameter of the cutting region 628 can accommodate the cutting device 626 within the lumen 610 in a non-actuated state, such that the cutting device 626 does not engage with the stent 614 (or other deliverable) disposed within the lumen 610. In some examples, not shown, the diameter of the lumen 610 is constant, including the diameter of the cutting region 628.

The cutting device 626 includes at least one cutting edge 630. Each cutting edge 630 is disposed at a free end of wedge-shaped projection extending from the surface of the cutting region 628 of the lumen 610. In some examples, as shown, the cutting device 626 includes two cutting edges 630. When the cutting device 626 is actuated, the two cutting edges 630 are drawn together and cooperate to cut an object positioned therebetween (e.g., stent 614) based on deformation of the inner shaft 606. In examples having one cutting edge 630, the cutting edge 630 can be drawn towards an opposing surface of the inner shaft 606, such that the single cutting edge 630 and the opposing surface cooperate to cut the object (e.g., stent 614) positioned therebetween.

The delivery apparatus 600 can be configured to selectively cut the stent 614 at the cutting edges 630, by actuating the cutting device 626. The cutting device 626 can be transitioned between a non-actuated state and an actuated state by translating the inner shaft 606 and the outer shaft 608 relative to each other. FIG. 8A illustrates the delivery apparatus 600 in the non-actuated state. In the non-actuated state, the stent 614 is moveable relative to the inner shaft 606 and the cutting edges 630 are spaced apart in the radial direction. For example, the stent 614 is able to move axially within the first lumen 610 and/or rotate within the first lumen 610 relative to the inner shaft 606 without engaging with the cutting device 626.

FIG. 8B illustrates the delivery apparatus 600 in the actuated state. In the actuated state, the cutting edge(s) 630 cooperate to cut the stent 614 therebetween based on deformation of the inner shaft 606 (e.g., similar to the locked state of delivery apparatus 100, 300, etc.). Specifically, as shown in FIG. 8B, the cutting device 626 is configured to cut the stent 614 into a distal portion 614d (e.g., distal to the cutting edges 630) and a proximal portion 614p (e.g., proximal to the cutting edges 630). The distal portion 614d of the stent 614 is the portion of the stent 614 that is implanted within a patient's vasculature 10 (e.g., within aneurysm 12) (FIG. 10). In some examples, in the actuated state, the proximal portion 614p of the stent 614 can be locked (e.g., immovable) relative to the inner shaft 606 (and therefore the delivery apparatus 600). For example, the proximal portion 614p of the stent 614 is not able to move axially within the first lumen 610 and/or rotate within the first lumen 610.

In the non-actuated state (FIG. 8A), the outer shaft 608 applies an axial force (e.g., no force, a nominal force, etc.) to the shoulder 618 that is insufficient to deform the inner shaft 606 (e.g., elongate the inner shaft 606 and compress the first lumen 610), such that the cutting edges 630 of the cutting device 626 remain spaced apart in the radial direction. As such, in the non-actuated state, the distal end 608d of the outer shaft 608 can either be spaced apart from the shoulder 618 or abut the shoulder 618 without applying sufficient axial force. For example, in some instances (e.g., when the outer shaft 608 abuts the shoulder 618 in the non-actuated state), a nominal force (e.g., a force less than axial force F_{A}) may be applied to the shoulder 618 that does not deform the inner shaft 606 to actuate the cutting device 626.

In the non-actuated state, the length of the inner shaft 606 is unstretched and the diameter of the first lumen 610 is uncompressed. As shown in FIG. 8A, the uncompressed diameter of the first lumen 610 (including the diameter of the cutting region 628) is greater than an outer diameter of the stent 614. In this way, the stent 614 can move (e.g., translate, rotate, etc.) relative to the inner shaft 606 within the first lumen 610 without engaging with the cutting device 626.

To transition from the non-actuated state (FIG. 8A) to the actuated state (FIG. 8B), the inner shaft 606 and the outer shaft 608 are translated relative to each other (e.g., by manipulating a handle of the delivery apparatus 600, etc.) along axis 605. In the actuated state, the outer shaft 608 applies a sufficient axial force F_{A} to the shoulder 618 to deform the inner shaft 606 (e.g., elongate the inner shaft 606 and compress the first lumen 610). When the first lumen 610 is compressed, the cutting edges 630 of the cutting device 626 are drawn together and cooperate to cut the stent 614 as described above. For example, the outer shaft 608 can be translated distally relative to the inner shaft 606 (or the inner shaft 606 can be translated proximally relative to the outer shaft 608) until the outer shaft 608 applies the sufficient axial force F_{A} to the shoulder 618, thus achieving the actuated state. As such, in the actuated state, the distal end 608d of the outer shaft 608 abuts or contacts the shoulder 618 while applying a sufficient axial force F_{A} to deform the inner shaft 606 and draw the cutting edges 630 together.

As shown in FIGS. 8A-8B, the cutting edges 630 are aligned in an axial direction (e.g., along axis 605). When the cutting device 626 is actuated to the actuated state, the cutting edges 630 are drawn together such that the cutting edges 630 contact each other. In some examples, not shown, the cutting edges 630 can be axially spaced apart, similar to a pair of scissors. In these examples, in the actuated state, the cutting edges 630 can contact each other and/or can be at least partially overlapped in a radial direction.

In the actuated state, the length of the inner shaft 106 is stretched or elongated and the diameter of the first lumen 610 is compressed. As described above, the magnitude of the axial force F_{A} can be proportional to the elongation of the inner shaft 606 and compression of the first lumen 610. As shown in FIG. 8B, the diameter of the cutting region 628 can be compressed, such that the cutting edges 630 are drawn together. In this way, in the actuated state, the inner shaft 606 applies a compressive, radial force F_{R} to the stent 614 via the cutting edges 630 to cut the stent 614 into the distal portion 614d and the proximal portion 614p.

As shown in FIG. 8B, the diameter of the remaining regions of the first lumen 610 can also be compressed, such that the first lumen 610 contacts the proximal portion 614p of the stent 614 (e.g., compresses against the proximal portion 614p). For example, the proximal portion 614p can limit the ability of the first lumen 610 to compress further in the radial direction. In this way, in the actuated state, the inner shaft 606 also applies the radial force F_{R} to the proximal portion 614p of the stent 614. The radial force F_{R} can be sufficient to lock the proximal portion 614p relative to the inner shaft 606 (e.g., such that the proximal portion 614p cannot move axially and/or rotationally relative to the inner shaft 606 within the first lumen 610). When the radial force F_{R} is applied, friction between the proximal portion 614p and the inner shaft 606 can prevent the relative movement therebetween. In some examples, the radial force F_{R} can be proportional to the axial force F_{A}.

As shown, the radial force F_{R} can be distributed along a length of the inner shaft 606, including along at least a portion of the cutting region 628 of the inner shaft 606. For example, the radial force F_{R} can be distributed (e.g., evenly distributed) along a portion of the inner shaft 606 comprising the same material properties (e.g., durometer hardness, tensile strength, etc.). In some examples, at least some of the radial force F_{R} can be localized within the cutting region 628 at the cutting device 626 to facilitate and/or improve the cutting of the stent 614, based on the geometry of the inner shaft 606 and the cutting device 626 and/or the material properties of the cutting region 628.

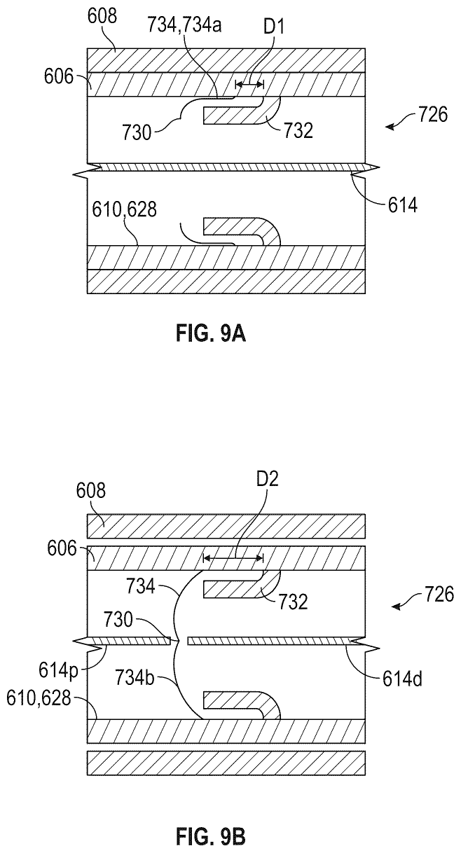

FIGS. 9A-9B illustrate an example cutting device 726 coupled to the inner shaft 606 and disposed within the first lumen 610 in lieu of cutting device 626. The cutting device 726 (e.g., a knife, a blade, a wedge, etc.) includes at least one cutting edge 730 and at least one flap 732 extending from the first lumen 610 (e.g., from the cutting region 628, etc). The cutting edges 730 are disposed at free ends of blades 734. In some examples, as shown, the cutting device 726 includes two blades 734 and two corresponding flaps 732. In some examples, not shown, the cutting device 726 can include one flap 732 extending the circumference of the inner lumen 610, regardless of the number of blades 734.

In the non-actuated state, the blades 734 are at least partially sheathed within the flaps 732, such that the cutting edges 730 are radially spaced apart from each other and the stent 614. Specifically, in the non-actuated state, a sheathed portion 734a of each blade 734 is disposed radially between the flap 732 and the surface defining the first lumen 610. In some examples, the entire blade 734 is sheathed in the non-actuated state. In some examples, as shown, a portion of the blade 734 can be unsheathed (e.g., an unsheathed portion 734b of the blade 734) in the non-actuated state. When the cutting device 726 is actuated, a greater portion of each blade 734 is unsheathed from the flaps 732, which permits the two cutting edges 630 to curve towards each other. As shown, the unsheathed portion 734b of the blade 734 can be disposed radially inwards of the flap 732 in the actuated state. When the blades 734 are sufficiently unsheathed, the cutting edges 730 can cooperate to cut an object positioned therebetween (e.g., stent 614). In examples having one blade 734, the cutting edge 730 can be drawn towards an opposing surface of the inner shaft 606, such that the single blade 734 and the opposing surface cooperate to cut the object (e.g., stent 614) positioned therebetween.

In some examples, the blades 734 can be formed from a shape-memory material (e.g., Nitinol, etc.) and can be shape-set in a curved configuration. In this way, when the blades 734 are sheathed (or retained) within the flaps 732, the sheathed portions 734a are in a generally flattened or straightened configuration (e.g., along the first lumen 610). As the blades 734 are unsheathed, the unsheathed portions 734b draw together and curve towards each other.

In some examples, as shown, the flaps 732 are distal to the blades 734. In other examples, the flaps 732 can be proximal to the blades 734. In either configuration, the blades 734 are configured to curve towards each other when unsheathed from the flaps 732. In some examples, the flaps 732 and the inner shaft 606 are integrally formed together as a single, monolithic piece. In some examples, the flaps 732 are formed separate from the inner shaft 606 and coupled thereto.

The amount of each blade 734 that is either sheathed (e.g., portion 734a) or unsheathed (e.g., portion 734b) is dependent on the amount of deformation of the inner shaft 606. As described above, the inner shaft 606 and the outer shaft 608 can move relative to each other between the non-actuated and actuated states to elongate the inner shaft 606 (and decrease the diameter of the lumen 610). As shown in FIGS. 9A-9B, the location where the blade 734 is fixed to the inner shaft 606 is axially offset from the location where the flap 732 extends from the first lumen 610. In the non-actuated state (FIG. 9A), the blade 734 and the flap 732 are offset from each other by a first (e.g., unstretched) distance D1. In the actuated state (FIG. 9B), the blade 734 and the flap 732 are offset by a second (e.g., stretched) distance D2 based on the elongation of the inner shaft 606. As shown, distance D2 is longer than distance D1. In this way, the elongation of the inner shaft 606 enables the blades 734 to be unsheathed from the flaps 732 and cut the stent 614 into the distal portion 614d and the proximal portion 614p.

FIG. 10 illustrates deployment of the stent 614 from the delivery apparatus 600 within an aneurysm 12 of a patient's vasculature 10. As introduced above, the stent 614 is a coil stent (e.g., an annular coil stent, a ganglion-shaped stent, etc.). When the stent 614 is advanced distally relative to the inner shaft 606 (e.g., such that the stent 614 is not retained within the inner shaft 606), the stent 614 can coil, for example, within the aneurysm 12. After a sufficient amount of the stent 614 is positioned within the aneurysm 12, a cutting device (any cutting device described herein, e.g., cutting device 626, cutting device 726, etc.) can be actuated to cut the stent 614. Specifically, the outer shaft 608 and the inner shaft 606 can be translated relative to each other, such that the outer shaft 608 elongates the inner shaft 606 by applying an axial force F_{A} to the shoulder 618 of the inner shaft 606 (see FIGS. 8B and 9B). As described above, this causes the cutting device to cut the stent 614 into a distal portion 614d that can be implanted within the aneurysm 12 and a proximal portion 614p that can be locked within the inner shaft 606. After the stent 614 is cut, the delivery apparatus 600 (and the proximal portion 614p of the stent 614) can be removed from the patient's vasculature 10.

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (e.g., by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient's vasculature. Moreover, the disclosed delivery approaches are not intended to be limited. Any of the prosthetic valves disclosed herein can be implanted using any of various delivery procedures and delivery devices known in the art.

### Additional Examples of the Disclosed Technology

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.
Example 1. A method of delivering a prosthetic implant, comprising: inserting a delivery apparatus into a patient's vasculature, wherein the delivery apparatus comprises a first shaft, a second shaft, and an inner component disposed within a lumen of the first shaft; moving the inner component and the first shaft relative to each other; and locking the inner component and the first shaft relative to each other by applying an axial force to a shoulder of the first shaft with the second shaft.
Example 2. The method of any example herein, particularly example 1, wherein locking comprises elongating a portion of the first shaft proximal to the shoulder and radially compressing the lumen of the first shaft.
Example 3. The method of any example herein, particularly either example 1 or example 2, wherein moving comprises axially moving the inner component and the first shaft relative to each other.
Example 4. The method of any example herein, particularly either example 1 or example 2, wherein moving comprises rotationally moving the inner component and the first shaft relative to each other.
Example 5. The method of any example herein, particularly any one of examples 1-4, wherein applying the axial force comprises translating the second shaft relative to the first shaft in a distal direction.
Example 6. The method of any example herein, particularly any one of examples 1-5, further comprising moving the inner component and the first shaft together relative to the patient's vasculature after locking the inner component and the first shaft.
Example 7. The method of any example herein, particularly example 6, wherein moving the inner component and the first shaft together comprises coupling the inner component to a prosthetic implant disposed within the patient's vasculature.
Example 8. The method of any example herein, particularly any one of examples 1-6, further comprising cutting, by a cutting device positioned within the lumen of the first shaft, the inner component into a first portion and a second portion by applying the axial force to the shoulder of the first shaft with the second shaft; and implanting the first portion of the inner component within the patient's vasculature, wherein locking the inner component and the first shaft relative to each other comprises locking the second portion of the inner component and the first shaft relative to each other.
Example 9. A method of delivering a prosthetic implant, comprising: inserting a delivery apparatus into a patient's vasculature; moving a tool within a lumen of a first shaft of the delivery apparatus relative to the first shaft, the first shaft having a first length and the lumen having a first diameter; and locking the tool within the lumen of the first shaft by moving a second shaft of the delivery apparatus relative to the first shaft in an axial direction, such that a length of the first shaft increases from the first length to a second, stretched length and a diameter of the lumen decreases from the first diameter to a second, compressed diameter.
Example 10. The method of any example herein, particularly example 9, wherein moving the second shaft relative to the first shaft comprises applying a distal force to a shoulder of the first shaft with the second shaft.
Example 11. The method of any example herein, particularly either example 9 or example 10, wherein locking the tool comprises radially compressing an inner surface defining the lumen against an outer surface of the tool.
Example 12. The method of any example herein, particularly any one of examples 9-11, further comprising removing the delivery apparatus and the tool from the patient's vasculature.
Example 13. The method of any example herein, particularly any one of examples 9-12, further comprising coupling the tool to a prosthetic implant disposed within the patient's vasculature; and after locking the tool within the lumen, translating the delivery apparatus and the prosthetic implant together relative to the patient's vasculature.
Example 14. A delivery apparatus for a prosthetic implant, comprising: a handle; a first shaft extending distally from the handle, the first shaft having a first lumen and a first durometer hardness; a second shaft disposed within the first lumen and movable relative to the first shaft, the second shaft having a second lumen and a second durometer hardness; and an inner component disposed within the second lumen, wherein the delivery apparatus is moveable between an unlocked state and a locked state, wherein in the unlocked state, the inner component is movable relative to the second shaft, wherein in the locked state, the inner component is locked relative to the second shaft, and wherein the first durometer hardness is greater than the second durometer hardness.
Example 15. The delivery apparatus of any example herein, particularly example 14, wherein the first durometer hardness is at least 1.5 times greater than the second durometer hardness.
Example 16. The delivery apparatus of any example herein, particularly either example 14 or example 15, wherein the first shaft has a first tensile strength, wherein the second shaft has a second tensile strength, and wherein the first tensile strength is greater than the second tensile strength.
Example 17. The delivery apparatus of any example herein, particularly example 16, wherein the first tensile strength is at least three times greater than the second tensile strength.
Example 18. The delivery apparatus of any example herein, particularly any one of examples 14-17, wherein in the unlocked state, the second shaft comprises a first, unstretched length and the second lumen comprises a first diameter, wherein the first diameter is greater than an outer diameter of the inner component.
Example 19. The delivery apparatus of any example herein, particularly example 18, wherein in the locked state, the second shaft comprises a second, stretched length and the second lumen comprises a second, compressed diameter, wherein the second, stretched length is greater than the first, unstretched length, and wherein the first diameter is greater than the second, compressed diameter.
Example 20. The delivery apparatus of any example herein, particularly any one of examples 14-19, wherein in the locked state, the second lumen contacts an outer surface of the inner component.
Example 21. The delivery apparatus of any example herein, particularly any one of examples 14-20, wherein the second shaft comprises a shoulder, wherein the shoulder is distal to a distal end of the first shaft.
Example 22. The delivery apparatus of any example herein, particularly example 21, wherein in the locked state, the distal end of the first shaft contacts the shoulder and applies an axial force to the shoulder.
Example 23. The delivery apparatus of any example herein, particularly any one of examples 14-22, wherein in the locked state, the second lumen applies a radial force to the inner component, wherein the radial force is distributed along a length of the inner component.
Example 24. The delivery apparatus of any example herein, particularly any one of examples 14-23, wherein in the unlocked state, the inner component is axially movable and rotationally moveable relative to the second shaft.
Example 25. The delivery apparatus of any example herein, particularly any one of examples 14-24, wherein in the locked state, the inner component is axially locked and rotationally locked relative to the second shaft.
Example 26. A delivery apparatus for a prosthetic implant, comprising: a handle; a first shaft extending distally from the handle, the first shaft comprising a first surface defining a first lumen; a second shaft extending distally from the handle, the second shaft disposed within the first lumen, the second shaft comprising a second surface defining a second lumen, wherein the first shaft and the second shaft are moveable relative to each other; and a cutting device disposed within the second lumen of the second shaft, wherein the cutting device is configured to actuate between a non-actuated state and an actuated state based on relative movement of the first shaft and the second shaft relative to the first shaft.
Example 27. The delivery apparatus of any example herein, particularly example 26, wherein the cutting device comprises a first cutting edge and a second cutting edge, wherein the first cutting edge and the second cutting edge are radially spaced apart from each other in the non-actuated state.
Example 28. The delivery apparatus of any example herein, particularly example 27, wherein the first cutting edge and the second cutting edge contact each other in the actuated state.
Example 29. The delivery apparatus of any example herein, particularly either example 27 or example 28, wherein the first cutting edge and the second cutting edge are axially aligned.
Example 30. The delivery apparatus of any example herein, particularly either example 27 or example 28, wherein the first cutting edge and the second cutting edge are axially spaced apart from each other.
Example 31. The delivery apparatus of any example herein, particularly example 30, wherein in the actuated state, the first cutting edge and the second cutting edge are at least partially overlapped in a radial direction.
Example 32. The delivery apparatus of any example herein, particularly any one of examples 26-31, wherein the second shaft comprises at least one flap extending from the second surface and disposed within the second lumen, wherein a sheathed portion of the cutting device is disposed radially between the flap and the second surface in the non-actuated state.
Example 33. The delivery apparatus of any example herein, particularly example 32, wherein the sheathed portion of the cutting device comprises the entire cutting device.
Example 34. The delivery apparatus of any example herein, particularly either example 32 or example 33, wherein an unsheathed portion of the cutting device is disposed radially inwards of the flap in the actuated state.
Example 35. The delivery apparatus of any example herein, particularly example 34, wherein the cutting device comprises a shape-memory material, wherein the unsheathed portion of the cutting device curves radially inward relative to the second lumen in the actuated state.
Example 36. The delivery apparatus of any example herein, particularly any one of examples 32-35, wherein an axial distance between the flap and the cutting device is longer in the actuated state than in the non-actuated state.
Example 37. The delivery apparatus of any example herein, particularly any one of examples 26-36, wherein the second shaft comprises a first, unstretched length in the non-actuated state and a second, stretched length in the actuated state.
Example 38. The delivery apparatus of any example herein, particularly example 37, wherein the second shaft comprises an outer surface defining a shoulder, wherein a distal end of the first shaft is positioned proximal to the shoulder.
Example 39. The delivery apparatus of any example herein, particularly example 38, wherein the distal end of the first shaft contacts the shoulder in the actuated state.
Example 40. The delivery apparatus of any example herein, particularly any one of examples 26-39, wherein the second lumen comprises a first diameter in the non-actuated state and a second, compressed diameter in the actuated state.
Example 41. The delivery apparatus of any example herein, particularly any one of examples 26-40, further comprising a stent disposed within the second lumen, wherein the stent is moveable relative to the second lumen in the non-actuated state.
Example 42. The delivery apparatus of any example herein, particularly example 41, wherein the cutting device is configured to cut the stent into a distal portion and a proximal portion, wherein the distal portion is configured to be implanted within a patient's vasculature.
Example 43. The delivery apparatus of any example herein, particularly any one of examples 26-42, wherein the delivery apparatus is sterilized.

The features described herein with regard to any example can be combined with other features described in any one or more of the other examples, unless otherwise stated. For example, any one or more of the features of method can be combined with any one or more features of another method. As another example, any one or more features of one delivery apparatus can be combined with any one or more features of another delivery apparatus.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

1. A method of delivering a prosthetic implant, comprising:
inserting a delivery apparatus into a patient's vasculature, wherein the delivery apparatus comprises a first shaft, a second shaft, and an inner component disposed within a lumen of the first shaft;
moving the inner component and the first shaft relative to each other; and
locking the inner component and the first shaft relative to each other by applying an axial force to a shoulder of the first shaft with the second shaft.

2. The method of claim 1, wherein locking comprises elongating a portion of the first shaft proximal to the shoulder and radially compressing the lumen of the first shaft.

3. The method of claim 1, wherein moving comprises axially moving the inner component and the first shaft relative to each other.

4. The method of claim 1, wherein moving comprises rotationally moving the inner component and the first shaft relative to each other.

5. The method of claim 1, wherein applying the axial force comprises translating the second shaft relative to the first shaft in a distal direction.

6. The method of claim 1, further comprising moving the inner component and the first shaft together relative to the patient's vasculature after locking the inner component and the first shaft.

7. The method of claim 6, wherein moving the inner component and the first shaft together comprises coupling the inner component to a prosthetic implant disposed within the patient's vasculature.

8. The method of claim 1, further comprising cutting, by a cutting device positioned within the lumen of the first shaft, the inner component into a first portion and a second portion by applying the axial force to the shoulder of the first shaft with the second shaft; and
implanting the first portion of the inner component within the patient's vasculature,
wherein locking the inner component and the first shaft relative to each other comprises locking the second portion of the inner component and the first shaft relative to each other.

9. A delivery apparatus for a prosthetic implant, comprising:
a handle;
a first shaft extending distally from the handle, the first shaft having a first lumen and a first durometer hardness;
a second shaft disposed within the first lumen and movable relative to the first shaft, the second shaft having a second lumen and a second durometer hardness; and
an inner component disposed within the second lumen,
wherein the delivery apparatus is moveable between an unlocked state and a locked state,
wherein in the unlocked state, the inner component is movable relative to the second shaft,
wherein in the locked state, the inner component is locked relative to the second shaft, and
wherein the first durometer hardness is greater than the second durometer hardness.

10. The delivery apparatus of claim 9, wherein the first durometer hardness is at least 1.5 times greater than the second durometer hardness.

11. The delivery apparatus of claim 9, wherein the first shaft has a first tensile strength, wherein the second shaft has a second tensile strength, and wherein the first tensile strength is greater than the second tensile strength.

12. The delivery apparatus of claim 11, wherein the first tensile strength is at least three times greater than the second tensile strength.

13. The delivery apparatus of claim 9, wherein in the unlocked state, the second shaft comprises a first, unstretched length and the second lumen comprises a first diameter, wherein the first diameter is greater than an outer diameter of the inner component.

14. The delivery apparatus of claim 9, wherein the second shaft comprises a shoulder, wherein the shoulder is distal to a distal end of the first shaft.

15. The delivery apparatus of claim 9, wherein in the locked state, the second lumen applies a radial force to the inner component, wherein the radial force is distributed along a length of the inner component.

16. The delivery apparatus of claim 9, wherein in the unlocked state, the inner component is axially movable and rotationally moveable relative to the second shaft.

17. A delivery apparatus for a prosthetic implant, comprising:
a handle;
a first shaft extending distally from the handle, the first shaft comprising a first surface defining a first lumen;
a second shaft extending distally from the handle, the second shaft disposed within the first lumen, the second shaft comprising a second surface defining a second lumen, wherein the first shaft and the second shaft are moveable relative to each other; and
a cutting device disposed within the second lumen of the second shaft, wherein the cutting device is configured to actuate between a non-actuated state and an actuated state based on relative movement of the first shaft and the second shaft relative to the first shaft.
